(19) United States
(12) Patent Application Publication
(10) Pub. No.: US 2009/0024154 A1
(43) Pub. Date: Jan. 22, 2009

(54) POWER SUPPLY USING TIME VARYING SIGNAL FOR ELECTROLYTICALLY DETACHING IMPLANTABLE DEVICE

(76) Inventors: Michael Williams, Oakland, CA (US); Mehran Bashiri, San Carlos, CA (US); Scott Merchel, Livermore, CA (US)

Correspondence Address: VISTA IP LAW GROUP LLP, 12930 Saratoga Avenue, Suite D-2, Saratoga, CA, 95070, US

(21) Appl. No.: 12/167,955

(22) Filed: Jul. 3, 2008

Related U.S. Application Data
(60) Provisional application No. 60/951,138, filed on Jul. 20, 2007.

(51) Int. Cl. A61M 29/00; A61B 18/04

(52) U.S. Cl. 606/191; 606/32

(57) ABSTRACT

A medical system comprises an implant assembly including an elongated pusher member, an implantable device (e.g., a vaso-occlusive device) mounted to the distal end of the pusher member, and an electrolytically severable joint disposed on the pusher member, wherein the implantable device detaches from the pusher member when the severable joint is severed, the medical system comprising an electrical power supply coupled to the implant assembly, and configured for conveying a time varying signal having net positive electrical energy to the severable joint to detach the implantable device from the pusher member.

### RELATED APPLICATION DATA

The present application claims the benefit under 35 U.S.C. §119 to U.S. Provisional Application No. 60/951,138, filed Jul. 20, 2007, the contents of which are incorporated herein by reference as though set forth in full.

### FIELD OF THE INVENTION

The invention relates generally to implantable devices (e.g., embolic coils, stents, and filters) having flexible electrolytic detachment mechanisms.

### BACKGROUND

Implants may be placed in the human body for a wide variety of reasons. For example, stents are placed in a number of different anatomical lumens within the body. They may be placed in blood vessels to cover vascular lesions or to provide patency to the vessels. Stents are also placed in biliary ducts to prevent them from kinking or collapsing. Grafts may be used with stents to promote growth of endothelial tissue within those vessels. As another example, vena cava filters can be implanted in the vena cava to catch thrombus sloughed off from other sites within the body and carried to the implantation site via the blood stream.

As still another example, vaso-occlusive devices are used for a wide variety of reasons, including for the treatment of intravascular aneurysms. An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality. Vaso-occlusive devices can be placed within the vasculature of the human body, typically via a catheter, either to block the flow of blood through a vessel making up that portion of the vasculature through the formation of an embolus or to form such an embolus within an aneurysm stemming from the vessel. The embolus seals and fills the aneurysm, thereby preventing the weakened wall of the aneurysm from being exposed to the pulsing blood pressure of the open vascular lumen.

One widely used vaso-occlusive device is a helical wire coil having windings, which may be dimensioned to engage the walls of the vessels. These coils typically take the form of soft and flexible coils having diameters in the range of 10-30 mils. Multiple coils will typically be deployed within a single aneurysm. There are a variety of ways of discharging vaso-occlusive coils into the human vasculature. In addition to a variety of manners of mechanically deploying vaso-occlusive coils into the vasculature of a patient, U.S. Pat. No. 5,122,136, issued to Guglielmi et al., describes an electrolytically detachable vaso-occlusive coil that can be introduced through a microcatheter and deployed at a selected location in the vasculature of a patient.

This vaso-occlusive coil is attached (e.g., via welding) to the distal end of an electrically conductive pusher wire. With the exception of a sacrificial joint just proximal to the attached embolic device, the outer surface of the pusher wire is coated with an ionically non-conductive material. Thus, the sacrificial joint will be exposed to bodily fluids when deployed within the patient. A power supply is used to provide direct current (DC) power to the core wire, with a conductive ground patch or intravenous needle located on or in the patient providing a ground return path. Applying a positive DC voltage to the pusher wire via the power supply relative to the ground return causes an electrolytic reaction between the sacrificial joint and the surrounding bodily fluid (e.g., blood). As a result, the sacrificial joint will dissolve, thereby detaching the vaso-occlusive coil from the pusher wire at the selected site.

While the use of electrolytically detachable vaso-occlusive coils has generally been successful, the period of time needed to detach the vaso-occlusive coils from the pusher wire is relatively long (currently, averaging from 30 to 40 seconds) and variable, resulting in an increase in procedure time. This problem is compounded by the need to deploy multiple vaso-occlusive coils within the patient. The relatively long and varying detachment time is due, in large part, to the relatively large and widely varying tissue impedance between the sacrificial joint and the ground electrode amongst patients. Many factors can affect the impedance at the detachment zone, including the formation of bubbles during the electrolytic process and the aggregation of blood constituents and electrolytic products. In addition, the bodily fluid surrounding the sacrificial joint may not be the optimum electrolyte (compared with saline) for inducing an electrolytic reaction in the detachment zone. It is believed that some blood constituents (proteins, fats, amino acids, etc.) may tend to aggregate into a cloud and/or adhere to the electrically active sacrificial joint which will tend to increase impedance, decrease the rate of electrolysis, thereby increasing the overall detachment time. Blood environment may also introduce variability in detachment time due to variations in blood constituents amongst patients. There, thus, remains a need to provide an improved electrolytic means for deploying implants within a patient.

### SUMMARY OF THE INVENTION

In accordance with one aspect of the present inventions, a medical system is provided. The medical system comprises an implant assembly including an elongated pusher member, an implantable device (e.g., a vaso-occlusive device) mounted to the distal end of the pusher member, and an electrolytically severable joint disposed on the pusher member. The implantable device detaches from the pusher member when the severable joint is severed. The medical system further comprises an electrical power supply coupled to the implant assembly. The power supply is configured for conveying a time varying signal having net positive electrical energy to the severable joint to detach the implantable device from the pusher member. The medical system may further comprise a delivery catheter configured for slidably receiving the implant assembly.

In one embodiment, the implant assembly further includes a terminal carried by the proximal end of the pusher member in electrical communication with the severable joint, wherein the terminal of the power supply is electrically coupled to the terminal of the implant assembly. In another embodiment, the power supply has another terminal electrically coupled to a return electrode, with the terminals of the power supply having different electrical potentials. The return electrode may be an external ground electrode or can be a return electrode carried by the pusher member. In one embodiment, the power supply is current-controlled (e.g., conveying the time varying signal in the range of 0.25 mA to 10 mA). In another embodiment, the power supply is voltage-controlled (e.g., conveying the time varying signal in the range of 0.5V to 11V). The time varying signal may have a suitable frequency (e.g., 10 Hz to 20 KHz). Although the present inventions should not be so limited in their broadest aspects, it is believed that the time varying signal allows the cloud of blood constituents that forms around the detachment site to dissipate, thereby decreasing and making the detachment time more consistent.

The time varying signal may take any of a variety forms (e.g., a square wave, a rounded square wave, a sinusoidal wave, or an exponential wave). The time varying signal may be a pulsed signal or a continuous signal. The time varying signal may have no portion that is negatively polarized or may have negatively polarized portions. In the latter case, the negatively polarized portions may additionally prevent buildup of the blood constituents and electrolysis products on the surface of the severable joint by temporarily reversing the electrical current. To the extent that the time varying signal has an effective duty cycle (i.e., the time period that the time varying signal is positively polarized divided by the total time period of the time varying signal), such duty cycle may be in a suitable range; for example, within the range of 5 percent to 95 percent, or within the range of 20 percent to 80 percent. The power supply may optionally be configured for amplitude modulating the time varying signal.

In accordance with another aspect of the present inventions, a method of implanting a medical device (e.g., a vaso-occlusive device) within a patient is provided. The method comprises introducing the medical device within the patient via a pusher member, and conveying a time varying signal having net positive electrical energy to a joint disposed on the pusher member to induce an electrolytic reaction at the joint, thereby severing the joint to detach the medical device from the pusher member at a target site (e.g., an aneurismal sac) within the patient. The method may further comprise introducing a delivery catheter within the patient, wherein the medical device is introduced within the patient via the delivery catheter, and removing the pusher member from the patient after medical device is detached from the pusher member.

In one method, the time varying signal is conveyed from the joint to a return electrode to induce the electrolytic reaction between the joint and the return electrode. The return electrode may be placed externally to the patient or may be carried by the pusher member. The time varying signal may be current-controlled (e.g., in the range of 0.25 mA to 10 mA) or voltage-controlled (e.g., in the range of 0.5V to 11V). The time varying signal may have a suitable frequency (e.g., in the range of 10 Hz to 20 KHz), and may take one of a variety of forms, such as those described above.

In accordance with a yet another aspect of the present inventions, a power supply is provided. The power supply comprises an electrical contact configured for being coupled to an implant assembly having a pusher member and an electrolytically detachable implantable device. The power supply further comprises power delivery circuitry configured for conveying a time varying signal having net positive electrical energy to the electrical contact to electrolytically detach the implantable device from the pusher member.

In one embodiment, the power supply further comprises another electrical contact configured for being coupled to a return electrode, which may be external or may be carried by the pusher member. The power delivery circuitry may be current-controlled (e.g., delivering the time varying signal in the range of 0.25 mA to 10 mA) or may be voltage-controlled (e.g., delivering the time varying signal in the range of 0.5V to 11V). The time varying signal may have a suitable frequency (e.g., in the range of 10 Hz to 20 KHz), and may take one of a variety of forms, such as those described above. The power supply may optionally comprise control circuitry configured for amplitude modulating the time varying signal.

Other and further aspects and features of the invention will be evident from reading the following detailed description of the preferred embodiments, which are intended to illustrate, not limit, the invention.

### BRIEF DESCRIPTION OF DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a plan view of a medical system arranged in accordance with one embodiment of the present invention, wherein the medical system particularly delivers a vaso-occlusive device into a patient using a bipolar electrolytic delivery means;

FIG. 2 is a plan view of a medical system arranged in accordance with another embodiment of the present inventions, wherein the medical system particularly delivers a vaso-occlusive device into a patient using a monopolar electrolytic delivery means;

FIGS. 3-11 are diagrams illustrating a variety of time-varying signals that can be generated by the power supply used in the medical systems of FIGS. 1 and 2 to electrolytically detach a vaso-occlusive device from a pusher member;

FIG. 12 is a block diagram of a power supply that can be used in either of the medical systems of FIGS. 1 and 2; and

FIGS. 13 and 14 are cross-sectional views illustrating a method of delivering a vaso-occlusive device within an aneurysm of the patient utilizing the medical systems of FIG. 1 or FIG. 2.

### DETAILED DESCRIPTION OF THE ILLUSTRATED EMBODIMENTS

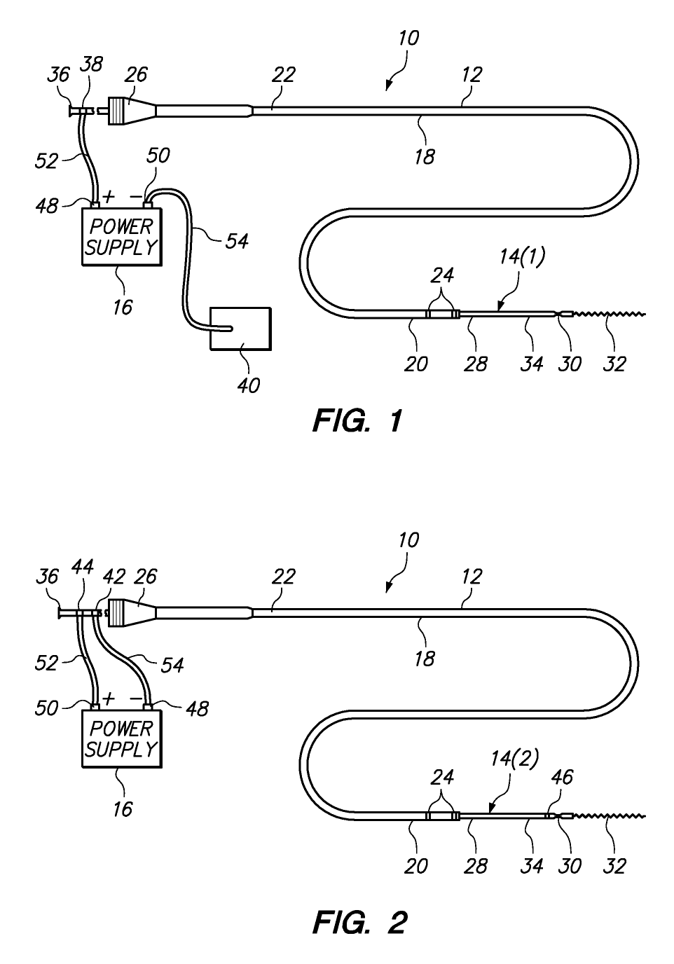

Referring generally to FIGS. 1 and 2, a medical system 10 constructed in accordance with one embodiment of the present inventions will be described. The medical system 10 is used in vascular and neurovascular indications, and particularly in the treatment of aneurysms, such as cerebral aneurysms. The medical system 10 utilizes an electrolytic detachment means to deploy vaso-occlusive devices, such as helical coils, within an aneurysm. Alternatively, the medical system 10 can be utilized to deploy implantable devices other than vaso-occlusive devices. For example, the medical system 10 can alternatively be used to deploy stents and vena cava filters, which are described in further detail in U.S. Pat. No. 6,468,266, which is expressly incorporated herein by reference.

To this end, the medical system 10 generally comprises a delivery catheter 12 that can be intravenously introduced within a patient to access a target site within the vasculature, an implant assembly 14 that can be slidably disposed within the delivery catheter 12, and an electrical power supply 16 that can supply electrical energy to the implant assembly 14 to effect the electrolytic detachment process.

The delivery catheter 12 includes an elongate, flexible, tubular member 28 composed of a suitable polymeric material and optionally reinforced with a coil or braid to provide strength or obviate kinking propensities. The delivery catheter 12 further includes a lumen (not shown) through which the implant assembly 14 can be selectively located. The delivery catheter 12 further includes a pair of radiopaque markers 24 disposed on the distal end 20 of the tubular member 28 to allow visualization of the delivery catheter 12 relative to the vaso-occlusive implant 22. The delivery catheter 12 further includes a proximal fitting 26 disposed on the proximal end 22 of the tubular member 28 for introduction of the implant assembly 14, as well as for the optional introduction of dyes or treatment materials.

The implant assembly 14 includes a pusher member 28, an electrolytically severable joint 30, and a vaso-occlusive device 32 that detaches from the distal end 34 of the pusher member 28 when the joint 30 is electrolytically severed. The pusher member 28 typically includes an electrically conductive core (not shown) that provides the pusher member 28 with the necessary tensile and columnar strength, an electrically insulative covering (not shown), and flexible coils (shown) that increase the flexibility of the pusher member 28 at its distal end. Two types of implant assemblies 14 are illustrated: (1) a monopolar implant assembly 14(1) (shown in FIG. 1), which uses monopolar electrolytic means to detach the vaso-occlusive device 32 from the pusher member 28 at the severable joint 30; and (2) a bipolar implant assembly 14(2) (shown in FIG. 2), which uses bipolar electrolytic means to detach the vaso-occlusive device 32 from the pusher member 28 at the severable joint 30.

The monopolar implant assembly 14(1) includes a positive terminal 38 (shown mated with the power supply 16) disposed on the proximal end 36 of the pusher member 28. The positive terminal 38 may simply be formed on the proximal end 36 of the pusher member 28 by exposing the underlying core wire. The positive terminal 38 serves as a positive terminal that is electrically coupled to the severable joint 30. In this case, the system 10 includes an external return electrode 40 in the form of a ground patch electrode or a ground needle, which can be placed into contact with the patient's tissue remote from the implant assembly 14(1). Thus, a monopolar patient circuit can be formed between the severable joint 30 at the distal end of the pusher member 28 and the return electrode 32 remotely located from the severable joint 30. An optional intermediate return electrode (not shown) can be carried by the distal end 34 of the pusher member 28 to enhance the monopolar patient circuit.

In contrast, the bipolar implant assembly 14(2) includes positive and negative terminals 42, 44 disposed on the proximal end 36 of the pusher member 28 (shown mated with the power supply 16), and a return (ground) electrode 46 carried by the distal end 34 of the pusher member 28 adjacent to the severable joint 30. The positive terminal 42 is electrically coupled to the severable joint 30, whereas the negative terminal 44 is electrically coupled to the return electrode 46. Thus, a bipolar patient circuit can be formed between the severable joint 30 and the return electrode 46 at the distal end 34 of the pusher member 28. In an optional embodiment, the power supply includes a third electrode (not shown), which is configured at the distal end 34 of the pusher member 28 near and preferably physically between the severable joint 30 and the return electrode 46. The third electrode is in electrical communication with terminal (not shown) on the proximal end of the pusher member 28. The third electrode has a high input impedance, so negligible amount of current flows through it, and can be used to monitor and maintain a specific voltage potential level between the severable joint 30 and the electrolyte solution in a voltage-controlled configuration (this third electrode is not necessary in current-controlled configurations). Current- and voltage-controlled configurations will be described in further detail below. Additionally, the third electrode may not be desired for voltage control if the system is electrochemically stable and predictable.

In either of the monopolar or bipolar arrangements, the severable joint 30 serves as an anode, and the external ground electrode 40 or return electrode 46 serves as a cathode. In both of the monopolar and bipolar arrangements, the positive terminals 38, 42 are typically electrically coupled to the severable joint 30 via a stainless steel, or otherwise electrically conductive, core wire (not shown) that extends within and provides the necessary tensile and columnar strength for the pusher member 28. In the monopolar arrangement, however, the entire proximal end of the pusher member 28 will typically be uninsulated to expose the underlying core wire, which will serve at the positive terminal 38. Further details discussing various exemplary constructions of monopolar and bipolar implant assemblies are disclosed in U.S. Provisional Patent No. 60/939,032, entitled “Electrolytically Detachable Implantable Device With Return Electrode,” which is expressly incorporated herein by reference.

The power supply 16 conveys electrical energy to the implant assembly 14 (and in particular, the severable joint 30) and returns electrical energy either from the ground electrode 40 or the implant assembly 14 (and in particular, the return electrode 46), to effect the electrolytic detachment of the vaso-occlusive implant 22. To the end, the power supply 16 has a positive electrical contact 48 configured to mate with the positive terminal 38 of the monopolar implant assembly 14(1) or the positive terminal 42 of the bipolar implant assembly 14(2) via a cable 52, and a negative electrical contact 50 configured to mate with the external ground electrode 40 or the negative terminal 44 of the bipolar implant assembly 14(2) via a cable 54. Notably, for the purposes of this specification, the terms “positive” and “negative” with respect to a terminal or electrical contact is relative and merely means that the positive terminal or electrical contact has a greater voltage potential than that of the negative terminal or electrical contact

In alternative embodiments, the positive terminal 38 of the monopolar implant assembly 14(1) is mated directly to the positive electrical contact 48 of the power supply 16 in the case of a monopolar arrangement, and the positive and negative terminals 42, 44 of the bipolar implant assembly 14(2) are mated directly to the respective positive and negative electrical contacts 48, 50 of the power supply 16. In an optional embodiment, the power supply 16 can be configured as a hybrid power supply that can be selectively operated either in a monopolar energy delivery mode by conveying electrical energy between the severable joint 30 of the monopolar implant assembly 14(1) and the external ground electrode 40 to detach the vaso-occlusive device 32 from the pusher member 28, and in a bipolar energy delivery mode by conveying electrical energy between the severable joint 30 and return electrode 46 of the bipolar implant assembly 14(2) to detach the vaso-occlusive device 32 from the pusher member 28. Further details on this hybrid power supply are disclosed in U.S. Patent Application Ser. No. 60/949,830, entitled Hybrid Power Supply for Electrolytically Detaching Implantable Device from Monopolar/Bipolar Pusher Wire, which is expressly incorporated herein by reference.

Significantly, rather than conveying the electrical energy to the implant assembly 14 in the form of a direct current (DC) electrical signal as done in prior art embodiments, the power supply 16 conveys electrical energy in the form of a time varying signal with net positive energy (i.e., the integral of the time varying signal is positive). By varying the signal conveyed from the power supply 16 to the implant assembly 14, it has been shown that the time required to effect electrolytic detachment of the vaso-occlusive device 32 is decreased and more consistent. It is believed that the lulls between the peaks of the time varying signal allow dissipation of the cloud formed by the blood constituents during the electrolytic process, thereby decreasing and making the detachment time more consistent. Of course, the net positive energy contained within the time varying signal ensures that a net electrical current will flow from the severable joint 30 to the external ground electrode 40 or return electrode 46 via the blood, thereby ensuring that an electrolytic reaction occurs to dissolve the severable joint 30.

In certain embodiments, the time varying signal may be a pulsed signal (i.e., a signal that is alternately pulsed “on” and “off”) at a specific frequency and duty cycle. The “on” periods of the time varying signal induce the electrolytic process to gradually dissolve the severable joint 30 on the pusher member 28, while the “off” periods of the time varying signal allow the high impedance cloud of blood constituents within the detachment zone to dissipate.

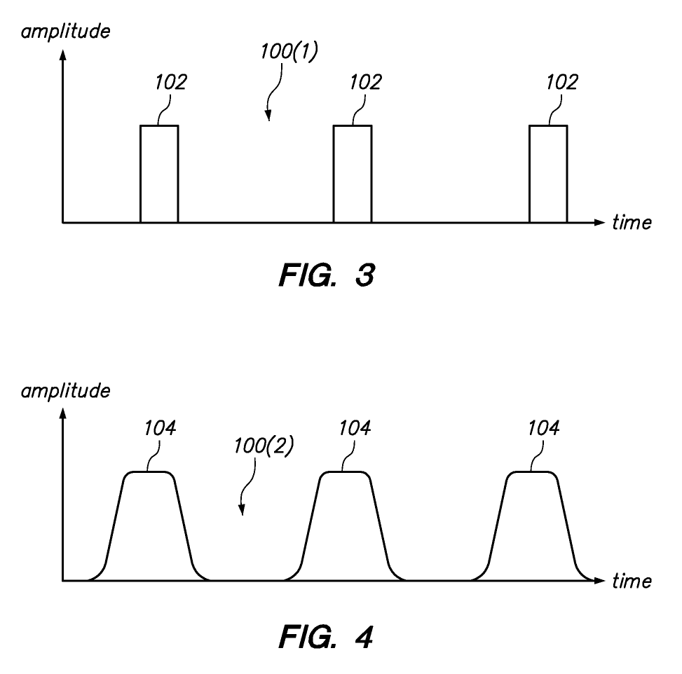

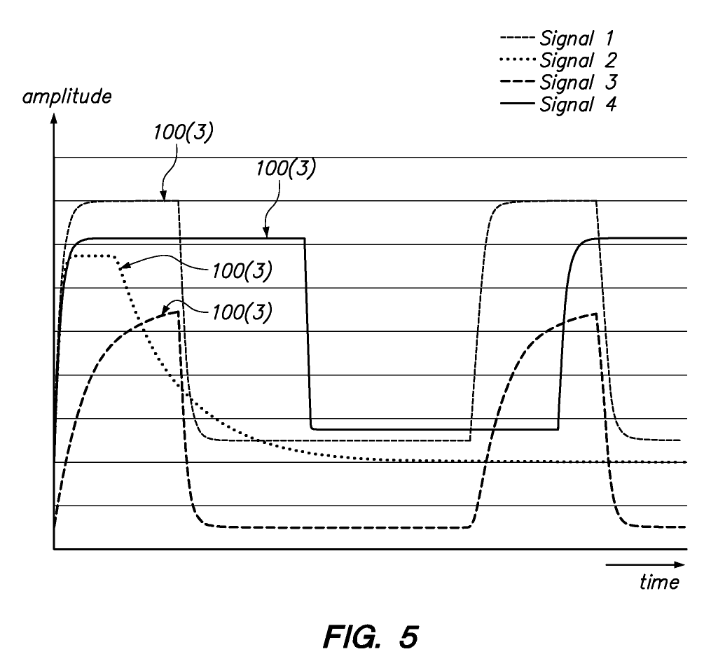

As one example, the time varying signal can take the form of a square wave 100(1), as illustrated in FIG. 3. As there shown, the square wave 100(1) includes positively polarized portions 102, each having a rectangular shape. In another example, the time varying signal can take the form of a rounded square wave 100(2), as illustrated in FIG. 4. As there shown, the rounded square wave 100(2) includes positively polarized portions 104, each having a rectangular shape with rounded corners. Notably, the rounded square wave 100(2) reduces the frequency content of the signal otherwise created by a pure square wave 100(1), thereby reducing the bandwidth requirements of the power supply 16. As another example, the time varying signal can take the form of any one of a variety of exponential waves 100(3), as illustrated in FIG. 5. The exponential waves 100(3) is similar to the round square wave 100(2) illustrated in FIG. 4, with the exception that each one exponentially increases to a high value and then exponentially decreases to a low value. As there shown, each exponential wave 100(3) includes positively polarized portions 106, each having a rounded leading edge 108 with an exponentially decreasing slope, and a lagging edge 110 with an exponentially decreasing slope. The different shapes between the various exponential waves 100(3) can be attributed to the different time constants used to characterize the waves. Notably, an exponential wave is similar to a rounded square wave in that it approximates the natural response of a real system with a pure square wave input. The advantages of an exponential wave is that it may initiate electrolytic reactions more steadily and consistently, compared to a pure square wave, and is easier on the electronics for maintaining signal output and monitoring, since the bandwidth of the electronics hardware required is lower.

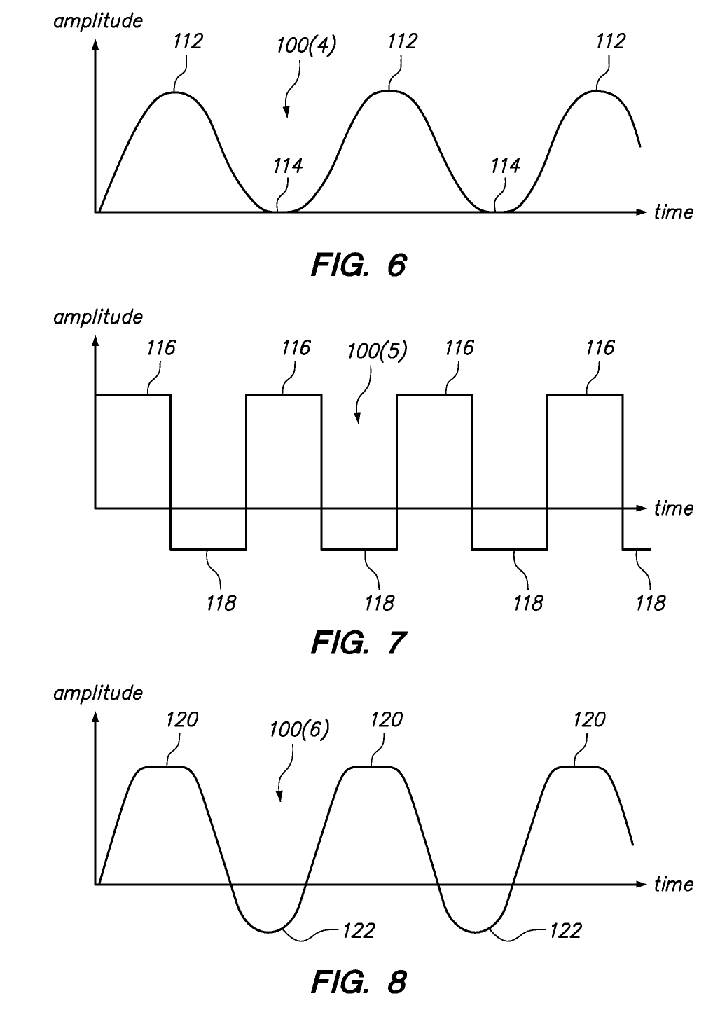

Although the time varying signals have been described as being pulsed, as illustrated in FIGS. 3-5, the time varying signal may be continuous. For example, the time varying signal may be an offset sinusoidal wave 100(4), as illustrated in FIG. 6. While the offset sinusoidal wave 100(4) does not have any “off” periods, the decreased electrical energy in the nulls 114 of the sinusoidal wave 100(4) facilitates dissipation of the blood constituent cloud between the peaks 112 of the sinusoidal wave 100(4).

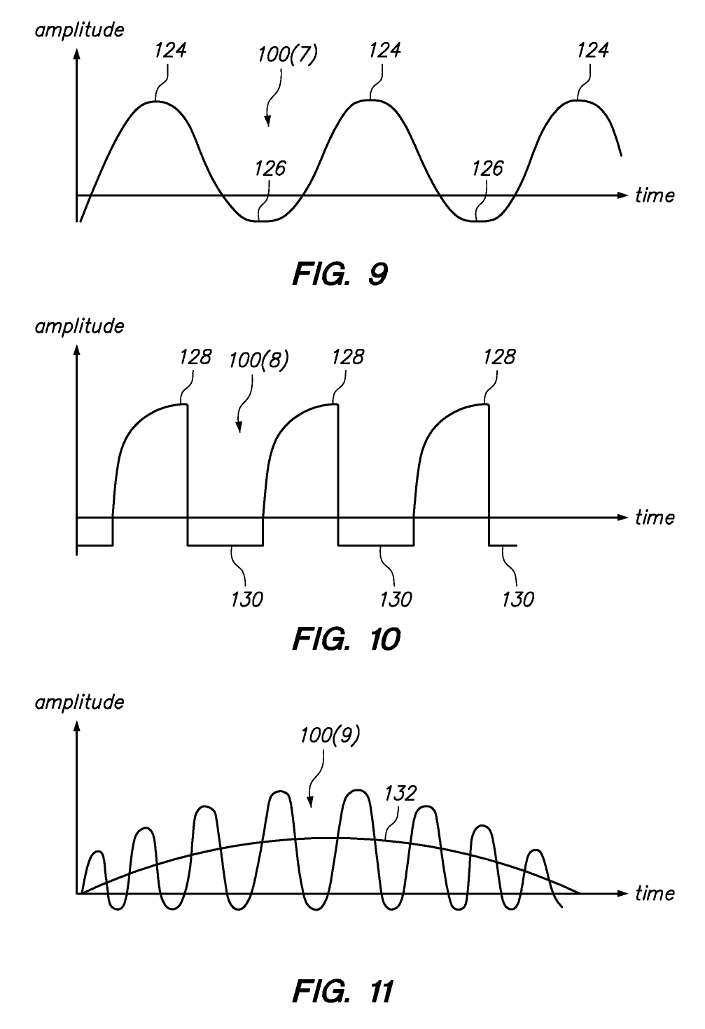

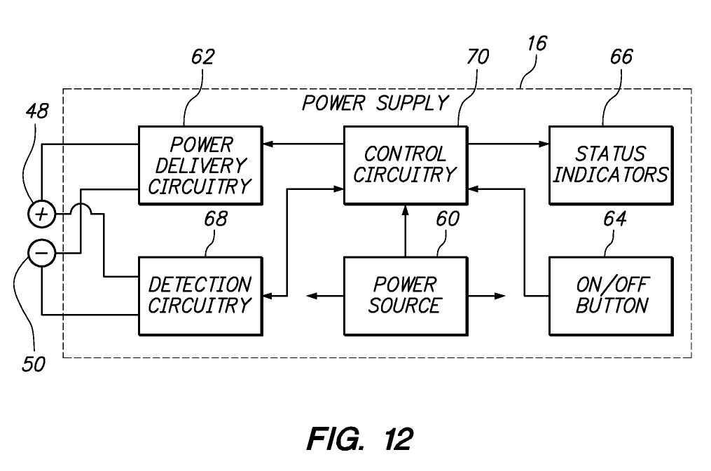

Although the previously described time varying signals have been described as having no negatively polarized portions, the time varying signal may have negatively polarized portions as long as the net energy of the time varying signal remains positive. For example, the time varying signal can take the form of an offset square wave 100(5) having positively polarized portions 116 and negatively polarized portions 118, as illustrated in FIG. 7. As another example, the time varying signal may take the form of an offset rounded square wave 100(6) having positively polarized portions 120 and negatively polarized portions 122, as illustrated in FIG. 8. As still another example, the time varying signal may take the form of an offset sinusoidal wave 100(7) having positively polarized portions 124 and smaller negatively polarized portions 126, as illustrated in FIG. 9. In yet another example, the time varying signal may take the form of an offset shark fin wave 100(8) having positively polarized portions 128 and smaller negatively polarized portions 130, as illustrated in FIG. 10.

As shown in FIGS. 7-10, the absolute magnitude of the positively polarized portions is greater than the absolute magnitude of the negatively polarized portions. As a result, the energy contained in the positively polarized portions is greater than the energy contained in the negatively polarized portions, so that the total net electrical energy remains positive. Notably, not only do the negatively polarized portions of the waveform facilitate dissipation of the blood constituent cloud (much like the “off” periods of the pulsed time period signals), the negatively polarized portions may actively disperse the aggregating/adhering blood constituents, bubbles, and electrolysis products (such as iron salts) on the surface of the severable joint 30 by temporarily reversing the electrical potential.

With the exception of the offset sinusoidal wave 100(4) illustrated in FIG. 6, which is continuously on and positively polarized, each of the illustrated time varying signals has an effective duty cycle, which for the purposes of this specification, is defined as the time period that the time varying signal is positively polarized divided by the total time period of the time varying signal. The effective duty cycle of the pulsed signal can be selected to optimize the induction of the electrolytic process for detachment against the dissipation of the blood constituent cloud and, in the case of a time varying signal having negatively polarized portions, removal of the blood constituents and electrolysis products on the several joint 30, to minimize the time necessary to completely dissolve the severable joint 30. The effective duty cycle may be in the range of 5% to 95%, preferably within the range of 20%-80%. The frequency of the time varying signal may be in a desired range, e.g., in the range of 10 Hz to 20 KHz.

In an optional embodiment, the power supply 16 is configured for amplitude modulating the time varying signal. For example, as illustrated in FIG. 11, the amplitude of the time varying signal, which in this case takes the form of a sinusoidal wave, may be gradually ramped up from the beginning to the middle of the electrolytic process, and then ramped down from the middle to the end of the electrolytic process, so that the magnitude of the electrical energy 132 is accordingly ramped up and then ramped down. In this manner, a small amount of energy is provided for initiation of the electrolysis process, and then a higher energy is provided for the continuation of the electrolysis process, while not creating bubbling. Thus, it can be appreciated that “shaping” the time varying signal may optimize the energy delivered for detachment during different stages of the electrolytic process as the severable joint 30 beings to pit and neck down.

Having described the function of the power supply 16, its components will now be described. The power supply 16 comprises a power source 60 configured for supplying power at the necessary voltage levels to the components of the power supply 16 and power delivery circuitry 62 configured for delivering the electrical energy necessary to electrolytically detach the vaso-occlusive device 32 of the implant assembly 14 coupled to the power supply 16.

The power source 60 may comprise conventional components, such as one or more batteries (e.g., standard 9V alkaline batteries or a AAA battery), and one or more voltage regulators (not shown) for converting the voltage provided by the output of the battery or batteries to different voltages that can be utilized by the components of the power supply 16.

The power delivery circuitry 62 may comprise an output drive circuit (not shown), which may take the form of a constant current source that will apply as much voltage as necessary to maintain the required current, a current-enable circuit (not shown) for turning the output drive circuit on, a current adjustment circuit (not shown) for adjusting the magnitude of the current output by the output drive circuit, and a patient isolation relay (not shown) that can be energized to decouple the implant assembly 14 from the output drive circuit during the power up diagnostics, after the vaso-occlusive device 32 is detached, or if a failure occurs during a procedure. In the illustrated embodiment, the current output by the power delivery circuitry 62 is a constant direct current (DC) waveform, although other waveforms that induce electrolysis can be used. The electrical energy conveyed from the power delivery circuitry 62 is preferably within the range of 0.1-10 milliamperes. If, alternatively, a voltage source is used, the electrical energy conveyed from the power delivery circuitry 62 is preferably within the range of 0.5-11 volts, preferably in the range of 4-8 volts.

The power supply 16 further comprises a power on/off actuator 64 configured for alternately activating and deactivating the power supply 16, and status indicators 66 for providing the status of the power supply 16 and electrolytic detachment process. The on/off actuator 64 may take the form of a conventional push button toggle switch that a user can alternately depress to activate and deactivate the power delivery circuitry 62. That is, initial actuation of the on/off actuator 64 will cause the power delivery circuitry 62 to deliver electrical energy to the mated implant assembly 14, and subsequent actuation of the on/off actuator 64 will cause the power delivery circuitry 62 to cease delivering electrical energy to the mated implant assembly 14. The status indicators 66 may take the form of any visible and/or audible indicators that provides status, such as low battery, power delivery state, detachment of the vaso-occlusive device 32, and misconnection within the patient circuit.

The power supply 16 further comprises detection circuitry 68 configured for detecting an electrical parameter indicative of a detachment event between the vaso-occlusive device 32 and the pusher member 28. In performing these functions, the detection circuitry 68 may comprises an alternating current (AC) signal generator (not shown) that superimposes or otherwise generates an AC signal in conjunction with the DC current generated the power delivery circuitry 62, and an AC-to-DC rectifier and peak detector (not shown) that measures the magnitude of the AC signal and outputs a DC signal. The detection circuitry 68 may also comprise a DC monitor for measuring the magnitude of the DC signal output by the power delivery circuitry 62.

The power supply 16 further comprises control circuitry 70 configured for monitoring and controlling the vital functions of the power supply 16. The control circuitry 70 may comprise a microcontroller that performs such functions, as controlling the current enable circuit of the power delivery circuitry in response to user operation of the on/off actuator 64, controlling the current adjust circuit and patient isolation relay of the power delivery circuitry under various conditions, determining detachment of the vaso-occlusive device 32 based on the feedback from the detection circuitry 68, managing the status indicators, running self-diagnostics, etc. The control circuitry 70 may be implemented in firmware, hardware, software, or in combination thereof. Alternatively, rather than using feedback to determine detachment, it can be assumed that the vaso-occlusive device 32 will predictably detach in a predetermined period of time in light of the improved detachment consistency provided by the previously described waveforms. In this case, the power supply can be verified/validated to effect detachment in a given period of time (e.g., 95% chance of detachment within 10 seconds).

With respect to the conventional functions performed by the power supply 16, much of the functional details of the foregoing components are described in U.S. Pat. Nos. 5,669,905 and 6,397,850, which are expressly incorporated herein by reference. However, as discussed above, the power supply 16 has the capability of delivering a time varying signal, which is optionally amplitude modulated, that minimizes and/or makes the electrolytic detachment time more consistent. The physical features of the power supply 16 may be similar to those described in U.S. Patent Application Ser. No. 60/949,830, entitled “Hybrid Power Supply for Electrolytically Detaching Implantable Device From Monopolar/Bipolar Pusher Wire,” which has been previously been expressly incorporated herein by reference.

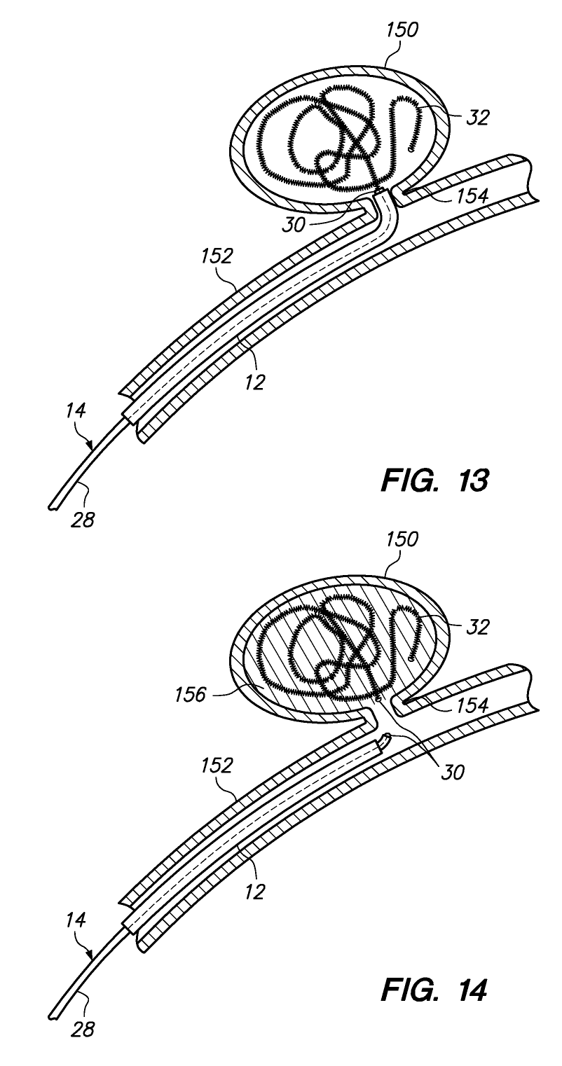

Having described the function and structure of the medical system 10, its operation in performing a medical procedure, and in particular implanting the vaso-occlusive device 32 within an aneurysm 150 of a patient, will now be described with reference to FIGS. 13 and 14. The process of implanting a vaso-occlusive device within a patient is typically practiced under fluoroscopic control with local anesthesia.

With reference to FIG. 13, the delivery catheter 12 is introduced within the patient via an entry point, such as the groin, and positioned within a blood vessel 152, with the tip of the catheter 12 residing within or adjacent a neck 154 of the aneurysm 150. The implant assembly 14 is then inserted within the delivery catheter 12 and advanced until the vaso-occlusive device 32 is disposed within the aneurysm 150. The implant assembly 14 is then coupled to the power supply 16. If the implant assembly 14 is monopolar, the ground electrode 40 will be coupled to the power supply 16, as illustrated in FIG. 1. If the ground electrode 40 takes the form of a patch electrode, it can be applied to the skin of the patient, e.g., on the shoulder. If the ground electrode 40 takes the form of a needle electrode, it can be percutaneously inserted into the patient, e.g., in the groin.

With reference to FIG. 14, electrical energy is delivered from the power supply 16 to the implant assembly 14 via actuation of the on/off power actuator 64 to severe the joint 30 at the distal end of the pusher member 28, thereby detaching the vaso-occlusive device 32 from the pusher member 28. The occlusion of the vaso-occlusive device 32 forms a coagulum 156 within the aneurysm 150, thereby eliminating the danger that the aneurysm 150 will rupture. Significantly, the electrical energy is delivered as a time varying signal, such as any of the ones illustrated in FIGS. 3-11, to decrease and make the detachment time more consistent.

In the case of a monopolar implant assembly 14(1) (shown in FIG. 1), the electrical energy will be delivered between the severable joint 30 and the ground electrode 40 to electrolytically detach the vaso-occlusive device 32 from the pusher member 28. In the case of a bipolar implant assembly 14(2) (shown in FIG. 2), the electrical energy will be delivered between the severable joint 30 and the return electrode 46 to electrolytically detach the vaso-occlusive device 32 from the pusher member 28. After the vaso-occlusive device 32 is detached, the pusher member 28 is removed from the delivery catheter 12. Additional vaso-occlusive devices may be implanted within the aneurysm 150 by introducing additional implant assemblies through the delivery catheter 12 and electrolytically detaching the vaso-occlusive devices within the aneurysm 150.

Although particular embodiments of the present inventions have been shown and described, it should be understood that the above discussion is not intended to limit the present inventions to these embodiments. It will be obvious to those skilled in the art that various changes and modifications may be made without departing from the scope thereof as defined by the claims.

1. A medical system, comprising:
an implant assembly including an elongated pusher member having a proximal end and a distal end, an implantable device mounted to the distal end of the pusher member, and an electrolytically severable joint disposed on the pusher member, wherein the implantable device detaches from the pusher member when the severable joint is severed; and
an electrical power supply coupled to the implant assembly, the power supply configured for conveying a time varying signal having net positive electrical energy to the severable joint to detach the implantable device from the pusher member.

2. The medical system of claim 1, wherein the implantable device comprises a vaso-occlusive device.

3. The medical system of claim 1, wherein the implant assembly further includes a terminal carried by the proximal end of the pusher member in electrical communication with the severable joint, wherein the terminal of the power supply is electrically coupled to the terminal of the implant assembly.

4. The medical system of claim 3, wherein the power supply has another terminal electrically coupled to a return electrode, the terminals of the power supply having different electrical potentials.

5. The medical system of claim 4, wherein the return electrode is carried by the distal end of the pusher member.

6. The medical system of claim 1, wherein the power supply is current-controlled.

7. The medical system of claim 6, wherein the amplitude of the time varying signal conveyed by the power supply is within the range of 0.25 mA to 10 mA.

8. The medical system of claim 1, wherein the power supply is voltage-controlled.

9. The medical system of claim 8, wherein the amplitude of the time varying signal conveyed by the power supply is within the range of 0.5V to 11V.

10. The medical system of claim 1, wherein the time varying signal has a frequency in the range of 10 Hz to 20 KHz.

11. The medical system of claim 1, wherein the time varying signal has an effective duty cycle within the range of 5 percent to 95 percent.

12. The medical system of claim 1, wherein the time varying signal has an effective duty cycle within the range of 20 percent to 80 percent.

13. The medical system of claim 1, wherein the time varying signal is a pulsed signal.

14. The medical system of claim 1, wherein the time varying signal is a continuous signal.

15. The medical system of claim 1, wherein no portion of the time varying signal is negatively polarized.

16. The medical system of claim 1, wherein the time varying signal has negatively polarized portions.

17. The medical system of claim 1, wherein the time varying signal is a square wave.

18. The medical system of claim 1, wherein the time varying signal is a rounded square wave.

19. The medical system of claim 1, wherein the time varying signal is a sinusoidal wave.

20. The medical system of claim 1, wherein the time varying signal is an exponential wave.

21. The medical system of claim 1, wherein the power supply is configured for amplitude modulating the time varying signal.

22. The medical system of claim 1, further comprising a delivery catheter configured for slidably receiving the implant assembly.

23. A method of implanting a medical device within a patient, comprising:
introducing the medical device within the patient via a pusher member; and
conveying a time varying signal having net positive electrical energy to a joint disposed on the pusher member to induce an electrolytic reaction at the joint, wherein the joint is severed to detach the medical device from the pusher member at a target site within the patient.

24. The method of claim 23, wherein the medical device comprises a vaso-occlusive device.

25. The method of claim 23, wherein the target site is an aneurismal sac.

26. The method of claim 23, further comprising conveying the time varying signal from the joint to a return electrode to induce the electrolytic reaction between the joint and the return electrode.

27. The method of claim 26, wherein the return electrode is carried by the distal end of the pusher member.

28. The method of claim 23, wherein the time varying signal is conveyed to the joint via the pusher member.

29. The method of claim 23, wherein the time varying signal is conveyed to the joint from a constant current source.

30. The method of claim 29, wherein the amplitude of the constant current source is within the range of 0.25 mA to 10 mA.

31. The method of claim 23, wherein the time varying signal is conveyed to the joint from a constant voltage source.

32. The method of claim 31, wherein the amplitude of the constant voltage source is within the range of 0.5V to 11V.

33. The method of claim 23, wherein the time varying signal has a frequency in the range of 10 Hz to 20 KHz.

34. The method of claim 23, wherein the time varying signal has an effective duty cycle within the range of 5 percent to 95 percent.

35. The method of claim 23, wherein the time varying signal has an effective duty cycle within the range of 20 percent to 80 percent.

36. The method of claim 23, wherein the time varying signal is a pulsed signal.

37. The method of claim 23, wherein the time varying signal is a continuous signal.

38. The method of claim 23, wherein no portion of the time varying signal is negatively polarized.

39. The method of claim 23, wherein the time varying signal has negatively polarized portions.

40. The method of claim 23, wherein the time varying signal is a square wave.

41. The method of claim 23, wherein the time varying signal is a rounded square wave.

42. The method of claim 23, wherein the time varying signal is a sinusoidal wave.

43. The method of claim 23, wherein the time varying signal is an exponential wave.

44. The method of claim 23, further comprising amplitude modulating the time varying signal.

45. The method of claim 23, further comprising introducing a delivery catheter within the patient, wherein the medical device is introduced within the patient via the delivery catheter.

46. The method of claim 23, further comprising removing the pusher member from the patient after medical device is detached from the pusher member.

47. A power supply, comprising:
an electrical contact configured for being coupled to an implant assembly having a pusher member and an electrolytically detachable implantable device; and
power delivery circuitry configured for conveying a time varying signal having net positive electrical energy to the electrical contact to electrolytically detach the implantable device from the pusher member.

48. The power supply of claim 47, further comprising another electrical contact configured for being coupled to a return electrode.

49. The power supply of claim 48, wherein the return electrode is carried by the distal end of the pusher member.

50. The power supply of claim 47, wherein the power delivery circuitry is current-controlled.

51. The power supply of claim 50, wherein the amplitude of the time varying signal delivered by the power delivery circuitry is within the range of 0.25 mA to 10 mA.

52. The power supply of claim 47, wherein the power supply includes a constant voltage source for conveying the time varying signal.

53. The power supply of claim 52, wherein the amplitude of the time varying signal delivered by the power delivery circuitry is within the range of 0.5V to 11V.

54. The power supply of claim 47, wherein the time varying signal has a frequency in the range of 10 Hz to 20 KHz.

55. The power supply of claim 47, wherein the time varying signal has an effective duty cycle within the range of 5 percent to 95 percent.

56. The power supply of claim 47, wherein the time varying signal has an effective duty cycle within the range of 20 percent to 80 percent.

57. The power supply of claim 47, wherein the time varying signal is a pulsed signal.

58. The power supply of claim 47, wherein the time varying signal is a continuous signal.

59. The power supply of claim 47, wherein no portion of the time varying signal is negatively polarized.

60. The power supply of claim 47, wherein the time varying signal has negatively polarized portions.

61. The power supply of claim 47, wherein the time varying signal is a square wave.

62. The power supply of claim 47, wherein the time varying signal is a rounded square wave.

63. The power supply of claim 47, wherein the time varying signal is a sinusoidal wave.

64. The power supply of claim 47, wherein the time varying signal is an exponential wave.

65. The power supply of claim 47, further comprising control circuitry configured for amplitude modulating the time varying signal.
